(19) United States
(12) Patent Application Publication
(10) Pub. No.: US 2018/0147073 A1
(43) Pub. Date: May 31, 2018

(54) LOWER LIMB ARTICULATION FOR BIPEDAL LOCOMOTION

(71) Applicants: UNIVERSITÉ DE BORDEAUX, Bordeaux (FR); INSTITUT POLYTECHNIQUE DE BORDEAUX, Talence Cedex (FR); CENTRE NATIONAL DE LA RECHERCHE SCIENTIFIQUE - CNRS, Paris Cedex 16 (FR); RHOBAN SYSTEM, Vayres (FR)

(72) Inventors: Olivier LY, Artigues-Près-Bordeaux (FR); Hugo GIMBERT, Bordeaux (FR); Grégoire PASSAULT, Bordeaux (FR); Quentin ROUXEL, Talence (FR)

(21) Appl. No.: 15/577,282

(22) PCT Filed: May 26, 2016
(86) PCT No.: PCT/FR2016/051243

§ 371 Date: Nov. 27, 2017

(30) Foreign Application Priority Data
May 27, 2015 (FR) 1554774

(51) Int. Cl. A61F 2/64; A61F 5/01; A61F 2/66; B25J 17/00

(52) U.S. Cl. CPC A61F 2/644; A61F 5/0123; A61F 2/646; A61F 2002/608; B25J 17/00; A61F 5/0127; A61F 2/6607

(57) ABSTRACT

Some embodiments are directed to a lower limb articulation for bipedal locomotion, which includes a first element a second element and articulation devices for articulating the first and second elements relative to one another, the articulation devices including an articulation mechanism including first and second linking devices mounted in series, as well as devices for guiding and locking in position one of the first and second elements relative to the other one of the first and second elements when the lower limb comes to rest on a surface, the devices for locking in position including a potential trough.

### CROSS REFERENCE TO RELATED APPLICATION(S)

This application is a national phase filing under 35 C.F.R. § 371 of and claims priority to PCT Patent Application No. PCT/FR2016/051243, filed on May 26, 2016, which claims the priority benefit under 35 U.S.C. § 119 of French Patent Application No. 1554774, filed on May 27, 2015, the contents of each of which are hereby incorporated in their entireties by reference.

### BACKGROUND

Some embodiments relate to a lower limb articulation for bipedal locomotion, in particular for a robot, or for serving as an orthosis for a patient.

In the related art, bipedal locomotion, performed in particular by robots of the humanoid type, is limited to almost level surfaces which offer practically no obstacle to movement. This is due in particular to the fact that the knee and ankle articulations of the lower limbs of such robots are designed around a single pivot connection. For example, the robots “ASIMO” and “HRP-4” incorporate harmonic speed reducers in order to minimize the operating clearance. This means, moreover, that these articulations are not reversible and can be set in motion by gravity only in an indirect manner.

The locomotion of such robots is without a blocking phase, for example without blocking of the knee articulation, on account inter alia of the nature of the motor primitives used excluding all singularities. Therefore, the result is that the supporting leg is constantly flexed, involving a very high motor torque in the motorization of the articulation.

### SUMMARY

Robots of the passive and semi-passive types include knee and ankle articulations designed around single pivot connections. However, these robots incorporate, in their walking primitive, a “blocked knee” phase. However, the purely passive robots can only move on an inclined plane free of any irregularities. For their part, the semi-passive robots are based on a motorization for performing a change of support and are in fact very sensitive to irregularities in the environment.

In orthoses type applications, the exoskeletons which permit compensation for muscle weakness of some patients and whose articulations are based on the preceding principles of the single pivotable connection, are bulky items of equipment and are limited in terms of energy autonomy and topography. Moreover, these exoskeletons are expensive.

Some embodiments are therefore related to a lower limb articulation for bipedal locomotion that is simple, compact and efficient, irrespective of the walking surface.

Some embodiments are directed to a lower limb articulation for bipedal locomotion, having a first element, a second element and articulation device for articulating the first and second elements relative to each other, the articulation device including an articulation mechanism having first and second connectors mounted in series, and also a device for guiding and blocking in position one of the first and second elements relative to the other one of the first and second elements when the lower limb comes to bear on a surface, the device for blocking in position having a potential well.

Advantageously, but optionally, the articulation according to some embodiments has at least one of the following additional technical features:
the first and second connectors are pivotable connectors which have a pivot axis orthogonal to a sagittal plane of the lower limb and which are mounted at a distance from each other;
the first connectors is a pivotable connectors which has a pivot axis orthogonal to a sagittal plane of the lower limb, and the second connectors is a connectors sliding in a direction parallel to the sagittal plane of the lower limb;
one of the first and second articulation devices has a travel-limiting system;
the other of the first and second connectors has a travel-limiting system;
the guiding and blocking device can include a cradle integral fixed to one of the first and second elements and arranged in such a way as to cooperate with a free end of the other one of the first and second elements;
the guiding and blocking device has a cam situated on the free end of the other one of the first and second elements and arranged in such a way as to come to bear against a surface of the cradle;
the cam includes a roller mounted freely in rotation on the free end of the other one of the first and second elements;
the cradle has an opening with an overall V shape or U shape in a sagittal plane of the lower limb;
the articulation is a knee articulation or ankle articulation.

Some embodiments are directed to a lower limb orthosis having at least one lower limb articulation with at least one of the above features.

### BRIEF DESCRIPTION OF THE FIGURES

Other features and advantages of the presently disclosed subject matter will become clear from the following description of embodiments according to the presently disclosed subject matter. In the attached drawings:

FIGS. 1a and 1b are schematics of a first embodiment of the lower limb articulation according to an embodiment of the presently disclosed subject matter;

FIG. 2 is a partial three-dimensional view of a first application, to the knee, of the embodiment of the presently disclosed subject matter from FIGS. 1a and 1b;

FIG. 3 is a partial three-dimensional view of a second application, to the ankle, of the embodiment of the presently disclosed subject matter from FIGS. 1a and 1b;

FIG. 4 is a partial three-dimensional view of a lower limb equipped with the articulations from FIGS. 2 and 3;

FIGS. 5a and 5b are principle schemes of a second embodiment of the lower limb articulation according to the presently disclosed subject matter;

FIG. 6 is a partial three-dimensional view of an application of the embodiment of the presently disclosed subject matter from FIGS. 5a and 5b; and

FIGS. 7a to 7d are diagrams illustrating a bipedal walking cycle with lower limb articulations according to an embodiment of the presently disclosed subject matter.

### DETAILED DESCRIPTION OF EMBODIMENTS

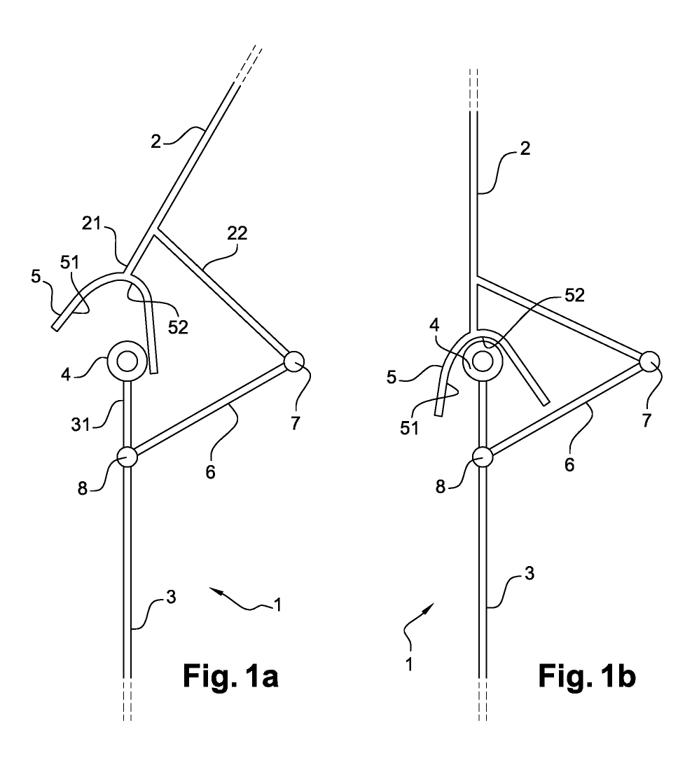

With reference to FIGS. 1a and 1b, we will describe a first embodiment of a lower limb articulation 1 according to the presently disclosed subject matter. In particular, we will describe the principle of the lower limb articulation 1 according to some embodiments. The lower limb articulation 1 according to some embodiments has a first element 2 which, in the context of a lower limb, may be the equivalent of the femur or of the foot, and a second element 3 which, in the context of a lower limb, may be the equivalent of the tibia/fibula. Moreover, the lower limb articulation 1 according to some embodiments has articulation devices 4, 5, 6, 7, 8 for articulating the first element 2 and second element 3 relative to each other.

The articulation device 4, 5, 6, 7, 8 includes an articulation mechanism 6, 7, 8. The articulation mechanism 6, 7, 8 has a first connector 7 and a second connector 8. The first connector 7 is a pivotable connector, of which a pivot axis is orthogonal to a sagittal plane of the lower limb having the lower limb articulation 1 according to some embodiments. The first pivotable connector 7 is positioned between an arm 22 of the first element 2, the arm 22 extending in the sagittal plane from an end 21 of the element 2 and toward the rear of the lower limb articulation 1 according to some embodiments, and one end of a connecting rod 6. The second connector 8 is a pivotable connector, of which a pivot axis is orthogonal to a sagittal plane of the lower limb having the lower limb articulation 1 according to some embodiments. The second pivotable connector 8 is positioned, for its part, between another end of the connecting rod 6 and the element 3, in the region of an end 31 of this element 3. Thus, the first 7 and second 8 pivotable connectors are mounted in series relative to each other, and at a distance from each other, their pivot axes being parallel with respect to each other. This permits an articulation mechanism that has two degrees of freedom.

Moreover, the articulation devices 4, 5, 6, 7, 8 include guiding and blocking devices 4, 5 for guiding and blocking one 2 of the first 2 and second 3 elements relative to the other 3 of the first 2 and second 3 elements when the lower limb including the lower limb articulation 1 according to some embodiments comes to bear on a walking surface, as will be explained later.

The guiding and blocking devices 4, 5 have a cradle 5. The cradle 5 is fixed to the end 21 of the first element 2. It includes an opening with what is generally a V shape or U shape, inverted in FIGS. 1a and 1b. The opening is in the sagittal plane of the lower limb including the lower limb articulation 1 according to some embodiments. The opening has an inner guiding surface 51 which includes a portion 52 forming a bottom. The cradle 5 is arranged in such a way as to cooperate with the end 31 of the second element 3. The end 31 is free. The guiding and blocking devices 4, 5 include a cam 4 positioned on the end 31 of the second element 3. In operation, this cam 4 is intended to come to bear on the inner guiding surface 51 of the cradle 5 and to slide along this surface toward the portion forming the bottom 52. In order to minimize friction, the cam 4 has a roller mounted freely in rotation on the end 31 of the second element 3. The roller can be a ball bearing, for example.

We will briefly describe an operation of the lower limb articulation 1 according to some embodiments. FIG. 1a illustrates a situation where the lower limb including the lower limb articulation 1 according to some embodiments is not bearing on a walking surface. The second element 3 is then free, and its end 31 is not necessarily in contact with the inner guiding surface 51 of the cradle 5, in particular with a portion of the inner guiding surface 51 located on one of the branches of the overall V shape or U shape of the opening. However, the cradle 5 can be arranged in such a way as to limit an amplitude of a movement in this situation.

FIG. 1b illustrates a situation where the lower limb including the lower limb articulation 1 according to some embodiments comes to bear on the walking surface. In this situation, the body weight transmitted by the first element 2 of the lower limb articulation 1 according to some embodiments causes a contact between the end 31 of the second element 3, via the cam 4, and the inner guiding surface 51 of the cradle 5. Therefore, the inner guiding surface 51 serves as a guide for the end 31 of the second element 3, and the lower limb articulation 1 according to some embodiments becomes taut, driven by the force exerted by the weight of the body under the effect of gravity. This brings the lower limb articulation 1 according to some embodiments to what is called a blocking position determined by the shape of the opening of the cradle 5: the cam 4 slides along the inner guiding surface 51 until it reaches the portion forming the bottom 52 of this surface. This portion forming the bottom 52 corresponds to a potential well for the lower limb articulation 1 according to some embodiments. More specifically, the contact between the cam 4 and the inner guiding surface 51 of the cradle permits the transmission of a force due to gravity in a direction orthogonal to a tangent to the inner guiding surface 51, at the point of contact with the cam 4. It is this force, thus generated, that brings the lower limb articulation 1 according to some embodiments to the blocking position as illustrated in FIG. 1b.

Thus, the force induced by gravity alone is sufficient to bring the lower limb articulation 1 according to some embodiments from a flexed position (FIG. 1a) to a blocking position (FIG. 1b). Accordingly, this change of position induces a horizontal displacement in the direction of walking (i.e. forward) of one end, opposite the end 21, namely the top end of the first element 2. Moreover, to a certain extent, the blocking position of the lower limb articulation 1 according to some embodiments, provided by the positioning of the cam 4 against the bottom portion 52 of the cradle 5, is effected independently of an initial amplitude of flexion of the lower limb including the lower limb articulation 1 according to some embodiments. Thus, the lower limb articulation 1 according to some embodiments operates irrespective of the irregularities in the walking surface. The force due to gravity in a direction orthogonal to a tangent to the inner guiding surface 51, at the point of contact with the cam 4, acts as a controller with regard to possible external disturbances to walking.

When the lower limb including the lower limb articulation 1 according to some embodiments departs from the position bearing on the walking surface, the two pivotable connectors 7, 8, which are now free, untension the lower limb articulation 1 according to some embodiments, which passes to a configuration similar to that illustrated in FIG. 1a.

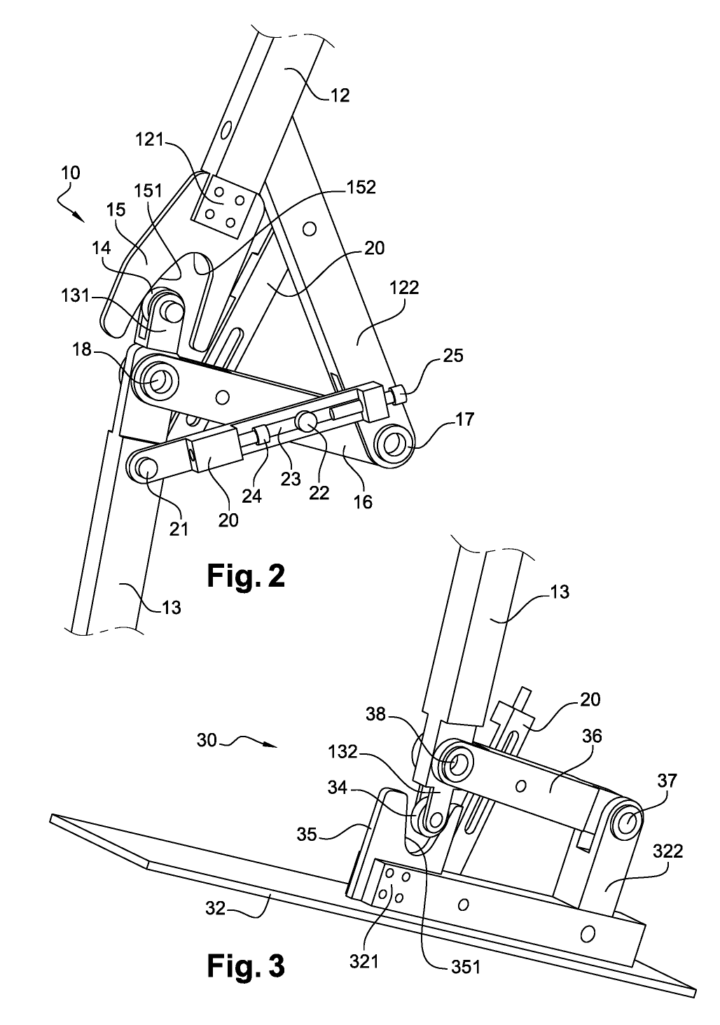

We will briefly describe a first application of this first embodiment of a lower limb articulation 1 according to some embodiments as just described. FIG. 2 illustrates a knee articulation 10 according to some embodiments. The first element 12 corresponds to the femur, and the second element 13 to the tibia/fibula. The end 121 of the femur 12 is rigidly fixed to the cradle 15, which includes an opening with an inner guiding surface 151 and a bottom portion 152. The tibia/fibula has a free end 131 on which a roller/cam is mounted freely in rotation. The articulation mechanism of the knee articulation 10 according to some embodiments has two pivotable connectors 17 and 18 similar to the previous pivotable connectors 7 and 8. The first pivotable connectors 17 is between an arm 122 of the femur 12, the arm 122 extending in the sagittal plane from the end 121 of the femur 12 and toward the rear of the knee articulation 10 according to some embodiments, and one end of a connecting rod 16. The second pivotable connectors 18 is for its part positioned between another end of the connecting rod 16 and the tibia/fibula 13, in the region of the end 131.

The operation is identical to the one explained above.

In order to limit a travel of the connectors 18, the knee articulation 10 according to some embodiments has a travel-limiting system 20. The travel-limiting system 20 is in the form of a bar including an oblong slot 23 on a first part. The bar is mounted pivotably 21 on the tibia/fibula 13 (for the pivotable connectors 18) and/or on the arm 122 (for the pivotable connectors 17). On the other part of the pivotable connectors 17, 18, the bar is mounted so as to slide along the oblong slot 23, via a pin 22 sliding in this oblong slot 23. The pin 23 is here positioned on the connecting rod 116, in both cases. Moreover, the travel-limiting system includes adjustment devices 24, 25 for adjusting a stroke of the pin 22 in the oblong slot 23. Here, the adjustment devices 24, 25 are in the form of grub screws.

FIG. 3 illustrates a second application of the first embodiment of a lower limb articulation 1 according to some embodiments described above. It is in this case an ankle articulation 30 according to some embodiments. The first element 32 corresponds to the foot, while the second element 13 is still the tibia/fibula. The foot 32 includes an end 321 including the cradle 35 having the inner guiding surface 351 similar to the inner guiding surfaces 51 and 151 described above. A second end 132 of the tibia/fibula 13 has a roller/cam 34 mounted freely in rotation, which cooperates with the cradle 35. The articulation mechanism includes two pivotable connectors 37, 38 separated by a connecting rod 36 and is similar to the articulation mechanisms 6, 7, 8 and 16, 17, 18 above. It is mounted between an arm 322 of the foot 32 and the end 132 of the tibia/fibula 13. Furthermore, the ankle articulation 30 according to some embodiments in this case has a single travel-limiting system 20 assigned to the pivotable connectors 37.

Again, the operation is identical to the one explained above.

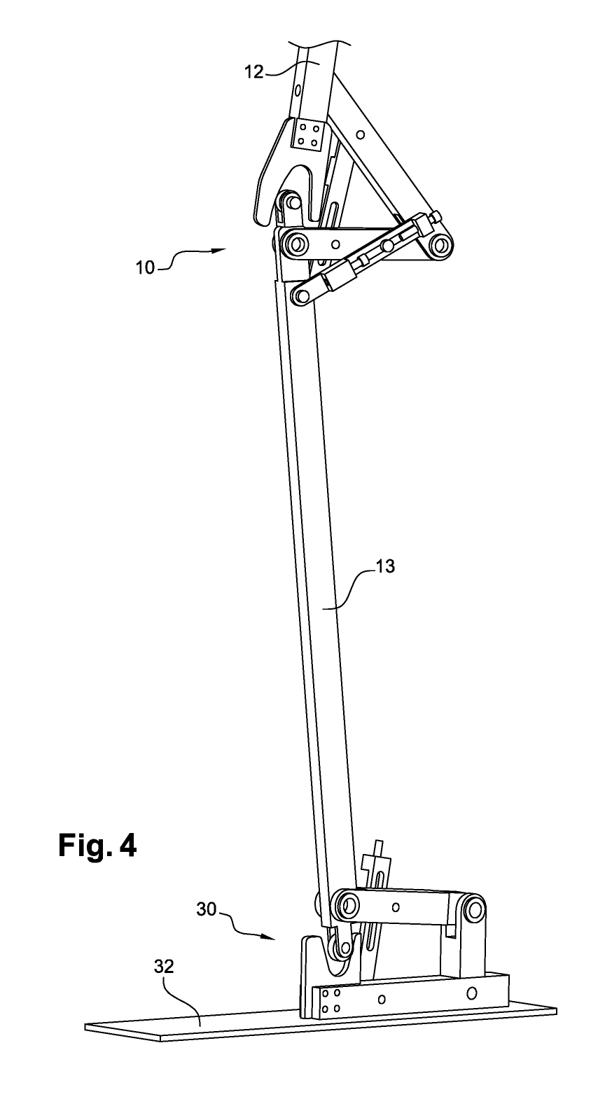

FIG. 4 illustrates a lower limb equipped with a knee articulation 10 and with an ankle articulation 30 according to some embodiments.

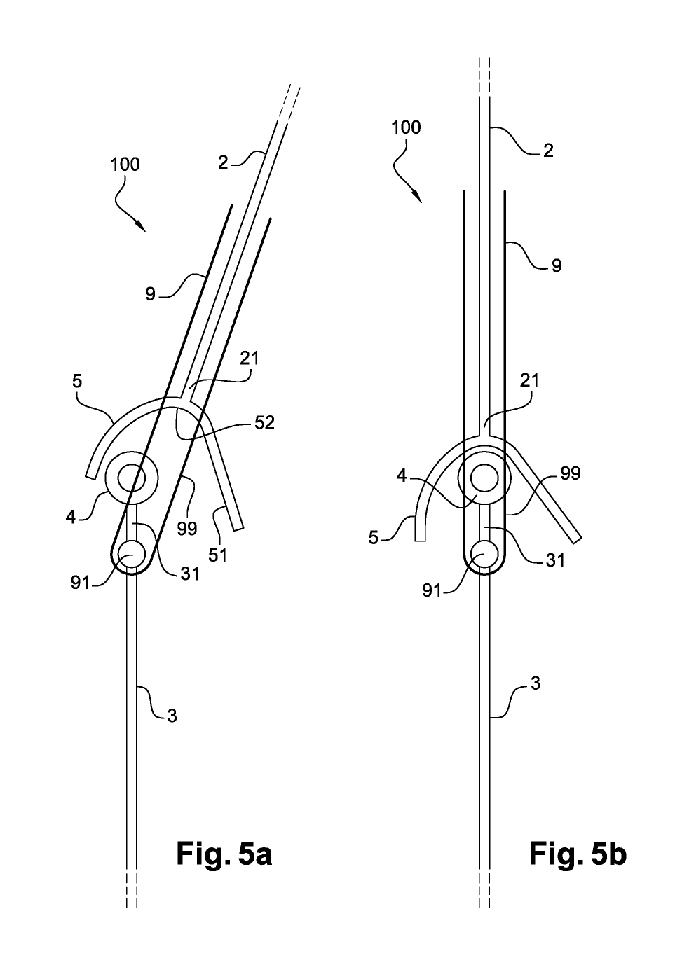

With reference to FIGS. 5a and 5b, we will now describe a second embodiment of a lower limb articulation 100 according to some embodiments. In particular, we will describe the principle of the lower limb articulation 100 according to some embodiments. The lower limb articulation 100 differs from the above-described lower limb articulation 1 according to some embodiments in terms of its articulation mechanism 9, 91. We will only describe the latter. The articulation mechanism 9, 91 has a first connector 91 and a second connector 9. The first connector 91 is a pivotable connector with a pivot axis orthogonal to the sagittal plane of the lower limb having the lower limb articulation 100 according to some embodiments. The first pivotable connector 91 is between the end 31 of the second element 3 and one end of a slide body 99. The second connector 9 is a slide connector in a direction parallel to the sagittal plane of the lower limb including the lower articulation limb 100 according to some embodiments. The second slide connector 9 is between the end 21 of the first element 2 and the slide body 99: the end 21 can slide in translation in the slide body 99. Again, the first 91 and second 9 connectors are mounted in series relative to each other. The operation of the lower limb articulation 100 is identical to that of the lower limb articulation 1 according to the presently disclosed subject matter of the first embodiment described above.

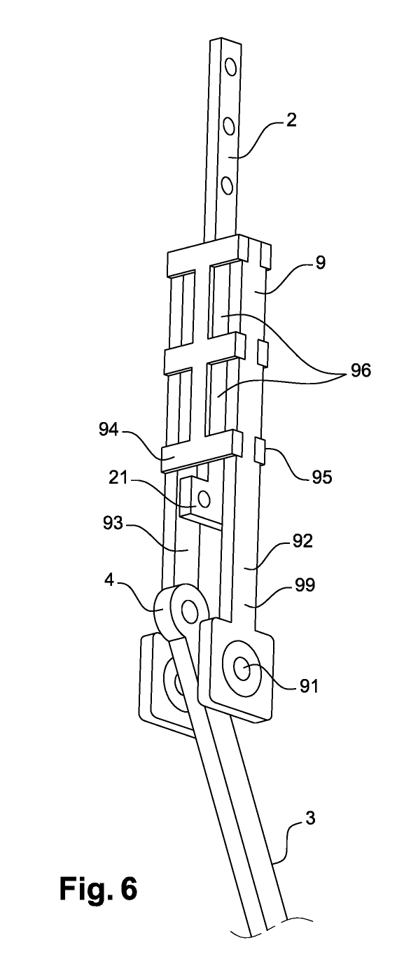

FIG. 6 illustrates an application of the articulation mechanism 9, 91 of the lower limb articulation 100 according to some embodiments. The slide body 99 has two flanks 92, 93 extending opposite each other and at a distance from each other. At a free end, the bottom end in the figure, the flanks 92, 93 have the first pivotable connector 91 with the second element 3. On an upper part in the figure, the slide body 99 has two crosspieces 94, 95 extending opposite each other and at a distance from each other. These crosspieces 94, 95 are fixed on the sides of the flanks 92, 93 and, with the latter, determine a volume in which are positioned some pads 96 which then surround a sliding rod of the first element 2. This rod carries the end 21 on which the cradle 5 (not shown in FIG. 6) is fixed. These pads 96 are arranged in such a way as to allow the rod to slide in the slide body in a longitudinal direction of this rod. This produces the second sliding connectors 9. The end 21 can then move in translation between the flanks 92, 93 opposite their lower parts in the figure. This structure makes it possible to integrate a travel-limiting system of the second sliding connectors 9: the travel is limited upwardly in the figure by the end 21 which has the cradle 5 coming to bear on the crosspieces 94, 95 and is limited downwardly by the contact between the cradle 5 and the roller/cam 4 of the second element 3.

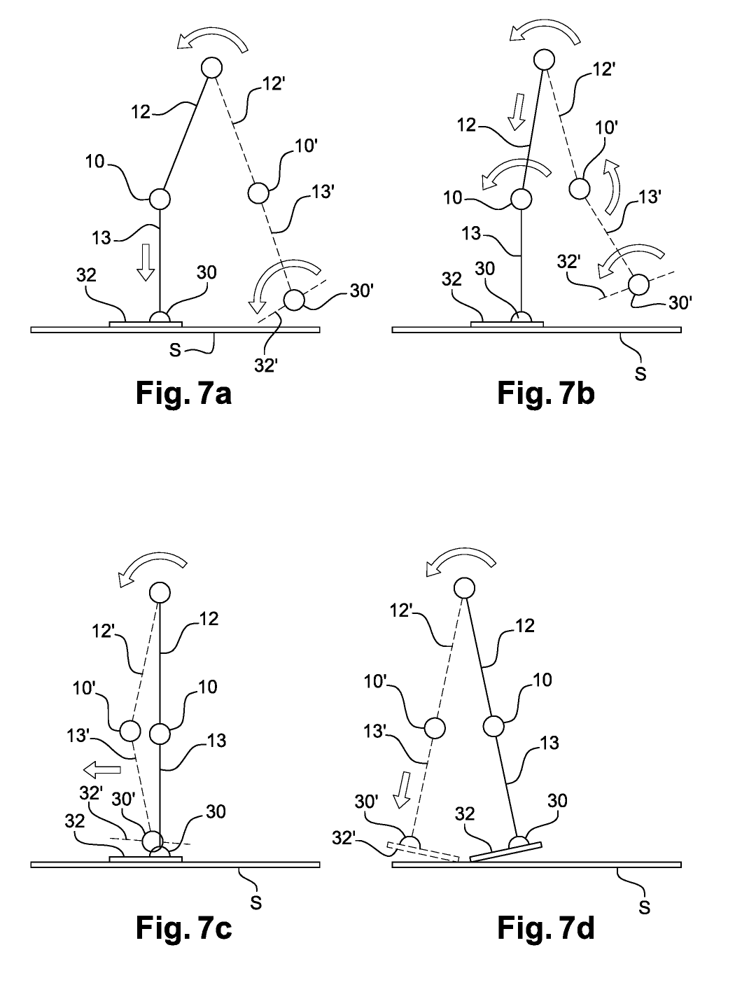

With reference to FIGS. 7a to 7d, we will now briefly describe a bipedal walking cycle that implements lower limb articulations according to some embodiments.

In phase 1 (FIG. 7a), the weight of the body is transferred from the rear lower limb (in broken lines) toward the front lower limb (in solid line lines). It is during this phase that energy is supplied to the system by a push on the rear ankle 30′. It should be noted that, in humans, this push is provided by the calf muscle, bearing on the toes. During this phase, the front ankle 30 is placed on the ground S, and its articulation 30 according to some embodiments is in the blocking phase as described above.

In phase 2 (FIG. 7b), the push of the ankle 30′ ceases, and the rear lower limb is no longer in a bearing position. Its knee articulation 10′ and ankle articulation 30′ are freed (situation then similar to that of FIGS. 1a and 4a in the embodiment of the articulations 10′ and 30′ according to the presently disclosed subject matter), and the rear lower leg adopts a flexed position, driven for example by return springs which can be provided for this purpose at the first and second connectors of the articulation mechanism of the articulations 10′ and 30′ according to some embodiments. Alternatively, this latter movement can also be produced by motors. It will be noted that, in this case, the motors in question then move only the lower limb that is not in a bearing position and, consequently, they require only a low torque and a reduced size.

In parallel, the front lower limb continues its blocking process by locking its knee articulation 10, and it does this without using motor force other than that caused by gravity acting on the body.

In phase 3 (FIG. 7c), the rear lower limb follows through in a pendulum movement causing it to move to the front. All the weight of the body is taken up by the lower limb “locked” in the straight position (knee articulation 10 blocked, likewise the ankle articulation 30).

In phase 4 (FIG. 7d), the supporting lower limb (solid lines) is unbalanced in a forward direction, rolling on the front of the foot 32, under the action of the movement and inertia of the body. The foot 32′ of the other lower limb (broken lines) rests on the ground S. It will be noted that the shock resulting from this contact is transmitted only to a lesser extent to the rest of the body on account of the free nature of the ankle articulation 30′ and knee articulation 10′. The cycle then begins again in phase 1.

Of course, it is possible to make many modifications to some embodiments without thereby departing from the scope of the latter.

1. A lower limb articulation for bipedal locomotion, comprising:
a first element;
a second element; and
an articulation device for articulating the first and second elements relative to each other, the articulation device including an articulation mechanism having first and second connectors mounted in series, and also a device for guiding and blocking in position one of the first and second elements relative to the other one of the first and second elements when the lower limb comes to bear on a surface, the device for blocking in position having a potential well.

2. The articulation as claimed in claim 1, wherein the first and second connectors are pivotable connectors which have a pivot axis orthogonal to a sagittal plane of the lower limb and which are mounted at a distance from each other.

3. The articulation as claimed in claim 1, wherein the first connector is a pivotable connector which has a pivot axis orthogonal to a sagittal plane of the lower limb, and the second connector is a connector sliding in a direction parallel to the sagittal plane of the lower limb.

4. The articulation as claimed in claim 2, one of a first and second articulation device has a travel-limiting system.

5. The articulation as claimed in claim 4, wherein the other of the first and second connectors has a travel-limiting system.

6. The articulation as claimed in claim 1, wherein the guiding and blocking device includes a cradle fixed to one of the first and second elements and arranged in such a way as to cooperate with a free end of the other one of the first and second elements.

7. The articulation as claimed in claim 6, wherein the guiding and blocking device has a cam situated on the free end of the other one of the first and second elements and arranged in such a way as to come to bear against a surface of the cradle.

8. The articulation as claimed in claim 7, wherein the cam includes a roller mounted freely in rotation on the free end of the other one of the first and second elements.

9. The articulation as claimed in claim 6, wherein the cradle has an opening with an overall V shape or U shape in a sagittal plane of the lower limb.

10. The articulation as claimed in claim 1, further including a knee articulation or an ankle articulation.

11. A lower limb orthosis, comprising: at least one articulation as claimed in claim 1.

12. The articulation as claimed in claim 3, wherein one of a first and second articulation device has a travel-limiting system.

13. The articulation as claimed in claim 2, wherein the guiding and blocking device includes a cradle fixed to one of the first and second elements and arranged in such a way as to cooperate with a free end of the other one of the first and second elements.

14. The articulation as claimed in claim 3, wherein the guiding and blocking device includes a cradle fixed to one of the first and second elements and arranged in such a way as to cooperate with a free end of the other one of the first and second elements.

15. The articulation as claimed in claim 4, wherein the guiding and blocking device includes a cradle fixed to one of the first and second elements and arranged in such a way as to cooperate with a free end of the other one of the first and second elements.

16. The articulation as claimed in claim 5, wherein the guiding and blocking device includes a cradle fixed to one of the first and second elements and arranged in such a way as to cooperate with a free end of the other one of the first and second elements.

17. The articulation as claimed in claim 7, wherein the cradle has an opening with an overall V shape or U shape in a sagittal plane of the lower limb.

18. The articulation as claimed in claim 8, wherein the cradle has an opening with an overall V shape or U shape in a sagittal plane of the lower limb.

19. The articulation as claimed in claim 2, further including a knee articulation or an ankle articulation.

20. The articulation as claimed in claim 3, further including a knee articulation or an ankle articulation.
